(19) United States
(12) Patent Application Publication
(10) Pub. No.: US 2012/0025954 A1
(43) Pub. Date: Feb. 2, 2012

(54) System and method for data management in automated biological instruments

(76) Inventors: Glenn Takayama, Danville, CA (US); Xiangchun Ye, San Ramon, CA (US); Samuel Burd, Oakland, CA (US); George Harter, Berkeley, CA (US)

(21) Appl. No.: 13/068,196

(22) Filed: May 4, 2011

Related U.S. Application Data
(62) Division of application No. 12/804,833, filed on Jul. 28, 2010.

(51) Int. Cl. G06K 7/01; C12M 1/00

(52) U.S. Cl. 340/10.1; 435/283.1; 901/27

(57) ABSTRACT

To simplify the process of preparing tissue specimens on slides, an advanced automated slide processing device is disclosed. The automated slide processing device comprises a robotic arm that both senses input and delivers output. The input sensing includes sensing radio frequency identifier tags (RFIDs) and the output includes writing to RFID tags. This allows the automated slide processing device to read RFID tags on chemical reagent containers, specimen slides, and slide processing modules. The information on the RFID tags can be used to program the automated slide processing device for a slide processing run, add new slide processing protocols, and determine what chemical reagents are available and where each chemical reagent containers is located.

### RELATED APPLICATIONS

This application is a divisional application from its parent application entitled “System And Method For Automated Tissue Slide Processing And Data Management” having Ser. No. 12/804,833 that was filed on Jul. 28, 2010.

### TECHNICAL FIELD

The present invention relates to the field of lab equipment. In particular the present invention discloses an automated system for processing slide-based tissue specimens and tracking the associated information.

### BACKGROUND

To properly examine tissues samples, most tissue samples are first processed with a defined protocol of slide preparation steps. For example, laboratories often apply stains to tissue slides in order to improve the contrast between individual parts of cells. The stains are absorbed differently by the various structures in cells such that the contrast between the different cell structures is greatly improved.

A modern laboratory that prepares and examines tissue specimens needs to be able to handle a wide variety of work. Many different types of tissues are handled, many different slide preparation protocols are used, and many different chemical reagents are used. And since the preparation and examination of tissues slides associated with medical tests can literally be a matter of life or death, all of these slide preparations must be carefully tracked and performed very accurately.

Processing tissue specimens and tracking the data is a meticulous task. Each slide with a tissue sample must be carefully tracked and put through a series of chemical processing and rinsing stages. Each chemical processing stage generally requires a specific amount of chemical reagent or buffer solution and takes a specific amount of time. Thus, trained technicians are generally employed to perform or supervise such operations.

To ensure that tissue slides can examined and compared on even basis, the tissue slide processing must be performed in a very consistent manner. Thus, it is very desirable to automate the chemical processing of tissue slides. By automating the chemical processing of tissue slides, expensive human labor is eliminated and the probability of an error occurring during the chemical processing of a tissue slide is greatly reduced. Furthermore, an automated tissue slide processing system will provide very consistent results due to very accurate measuring of reagents, very precise processing times, and the overall consistency provided any automated system. Thus, the use of automated slide processing systems has become very common within modern biological laboratories.

The existing set of automated tissue slide processing systems has greatly improved the quality of tissue slide preparation. However, there is still plenty of room for improvement. For example, automated tissue slide processing systems are still not very simple to use and an operator must be very careful in order to ensure that each slide is processed properly. Thus, it would be desirable to simplify the operation of an automated tissue slide processing system and improve the data tracking abilities of such a device.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a diagrammatic representation of the machine in the example form of a computer system within which a set of instructions for causing the machine to perform any one or more of the methodologies discussed herein may be executed.

FIG. 2A illustrates a top view of a slide processing apparatus having a set of slide modules holding glass slides, a set of reagent containers storing chemical reagents, and a robotic arm with a reagent dispenser for extracting chemical reagents from the reagent containers and depositing on the glass slides in the slide modules.

FIG. 2B illustrates a second embodiment of a slide processing apparatus with a different reagent container and slide module layout.

FIG. 3A illustrates a top down view of one embodiment of a reagent container that may be used within the reagent container storage area of the slide processing apparatus illustrated in FIG. 2A.

FIG. 3B illustrates a side view of one embodiment of a reagent container that may be used within the reagent container storage area of the slide processing apparatus illustrated in FIG. 2A.

FIG. 3C illustrates a view of the reagent container of FIG. 3B when it is allowed to rest on a long bottom surface of the container.

FIG. 3D illustrates an alternate embodiment of a reagent container that includes a wall around the bottom of the container to allow the container to stand vertically. [I put in both embodiments.]

FIG. 4A illustrates a top down view of one embodiment of a slide module that may be used within the slide module area of the slide processing apparatus illustrated in FIG. 2A.

FIG. 4B illustrates a side view of one embodiment of a slide module that may be used within the slide module area of the slide processing apparatus illustrated in FIG. 2A.

FIG. 4C illustrates the slide module of FIG. 4B wherein the chamber unit has been lifted up from the glass slide.

FIG. 5A illustrates a first example heating profile for use within a slide processing protocol.

FIG. 5B illustrates a second example heating profile for use within a slide processing protocol.

FIG. 5C illustrates an example of a chamber unit control and heating profile for use within a slide processing protocol.

FIG. 6 conceptually illustrates a head assembly of a robotic delivery arm situated above a set of reagent containers.

FIG. 7 illustrates a flow diagram that describes an example operation of the slide processing apparatus in one particular embodiment.

### DETAILED DESCRIPTION

The following detailed description includes references to the accompanying drawings, which form a part of the detailed description. The drawings show illustrations in accordance with example embodiments. These embodiments, which are also referred to herein as “examples,” are described in enough detail to enable those skilled in the art to practice the invention. It will be apparent to one skilled in the art that specific details in the example embodiments are not required in order to practice the present invention. For example, although some of the example embodiments are disclosed with reference to a slide processing system, the teachings can be used in many other environments. Thus, any digital system that uses digital memory can benefit from the teachings of the present disclosure. The example embodiments may be combined, other embodiments may be utilized, or structural, logical and electrical changes may be made without departing from the scope of what is claimed. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive or, such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Computer Systems

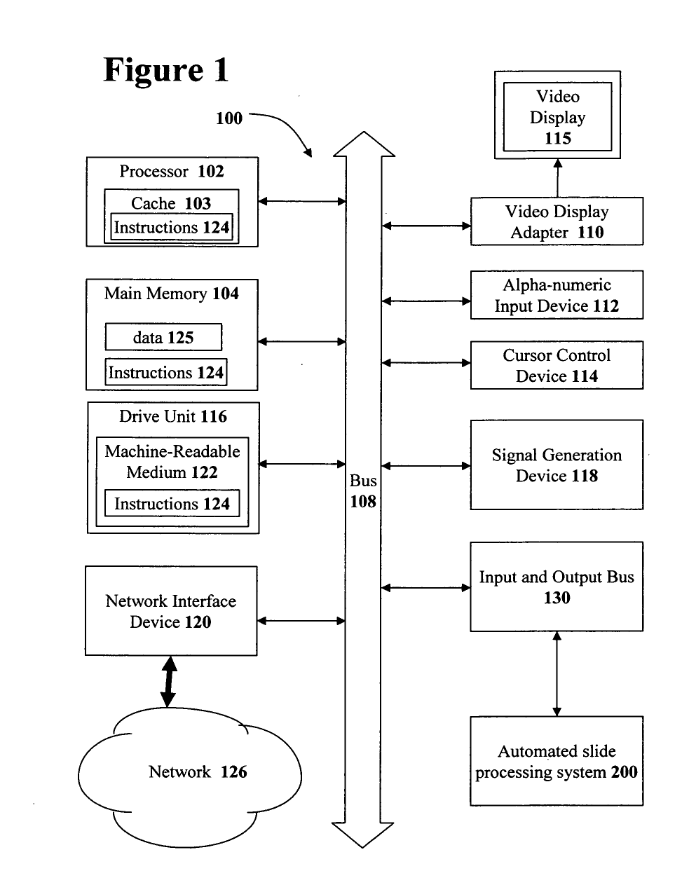

The present disclosure concerns digital computer systems. FIG. 1 illustrates a diagrammatic representation of a machine in the example form of a computer system 100 that may be used to implement portions of the present disclosure. Within computer system 100 of FIG. 1, there are a set of instructions 124 that may be executed for causing the machine to perform any one or more of the methodologies discussed within this document.

In a networked deployment, the machine of FIG. 1 may operate in the capacity of a server machine or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network server, a network router, a network switch, a network bridge, or any machine capable of executing a set of computer instructions (sequential or otherwise) that specify actions to be taken by that machine. Furthermore, while only a single machine is illustrated, the term “machine” shall also be taken to include any collection of machines that individually or jointly execute a set (or multiple sets) of instructions to perform any one or more of the methodologies discussed herein.

The example computer system 100 of FIG. 1 includes a processor 102 (e.g., a central processing unit (CPU), a graphics processing unit (GPU) or both) and a main memory 104, which communicate with each other via a bus 108. The computer system 100 may further include a video display adapter 110 that drives a video display system 115 such as a Liquid Crystal Display (LCD) or a Cathode Ray Tube (CRT). The computer system 100 also includes an alphanumeric input device 112 (e.g., a keyboard), a cursor control device 114 (e.g., a mouse or trackball), a disk drive unit 116, a signal generation device 118 (e.g., a speaker) and a network interface device 120. Note that not all of these parts illustrated in FIG. 1 will be present in all embodiments. For example, a computer server system may not have a video display adapter 110 or video display system 115 if that server is controlled through the network interface device 120.

The disk drive unit 116 includes a machine-readable medium 122 on which is stored one or more sets of computer instructions and data structures (e.g., instructions 124 also known as ‘software’) embodying or utilized by any one or more of the methodologies or functions described herein. The instructions 124 may also reside, completely or at least partially, within the main memory 104 and/or within a cache memory 103 associated with the processor 102. The main memory 104 and the cache memory 103 associated with the processor 102 also constitute machine-readable media.

The instructions 124 may further be transmitted or received over a computer network 126 via the network interface device 120. Such transmissions may occur utilizing any one of a number of well-known transfer protocols such as the well known File Transport Protocol (FTP).

While the machine-readable medium 122 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies described herein, or that is capable of storing, encoding or carrying data structures utilized by or associated with such a set of instructions. The term “machine-readable medium” shall accordingly be taken to include, but not be limited to, solid-state memories, optical media, and magnetic media.

For the purposes of this specification, the term “module” includes an identifiable portion of code, computational or executable instructions, data, or computational object to achieve a particular function, operation, processing, or procedure. A module need not be implemented in software; a module may be implemented in software, hardware/circuitry, or a combination of software and hardware.

Automated Slide Processing System Overview

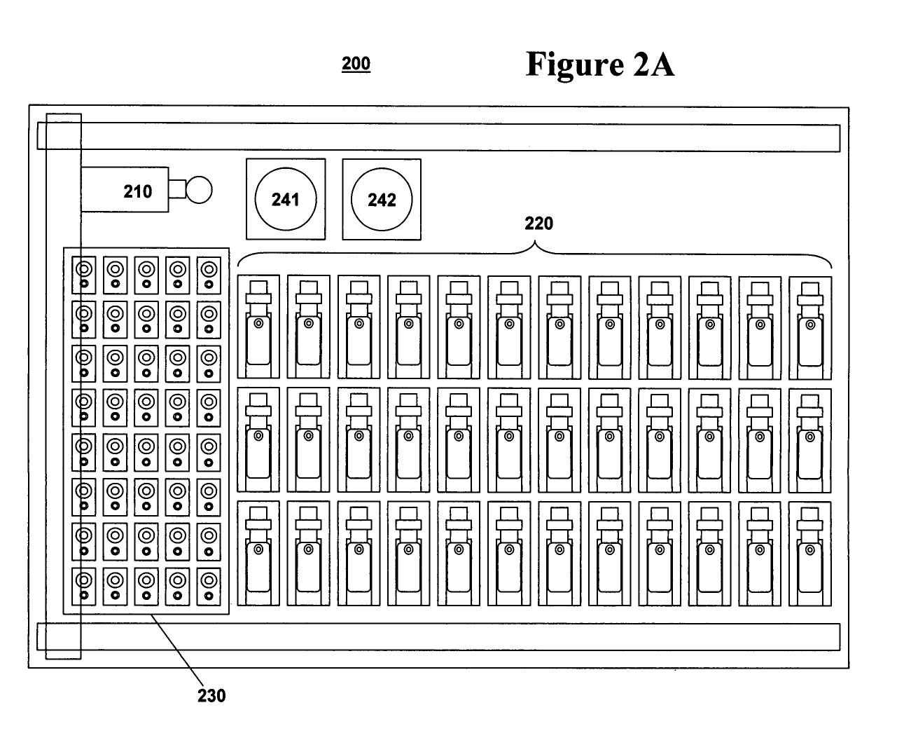

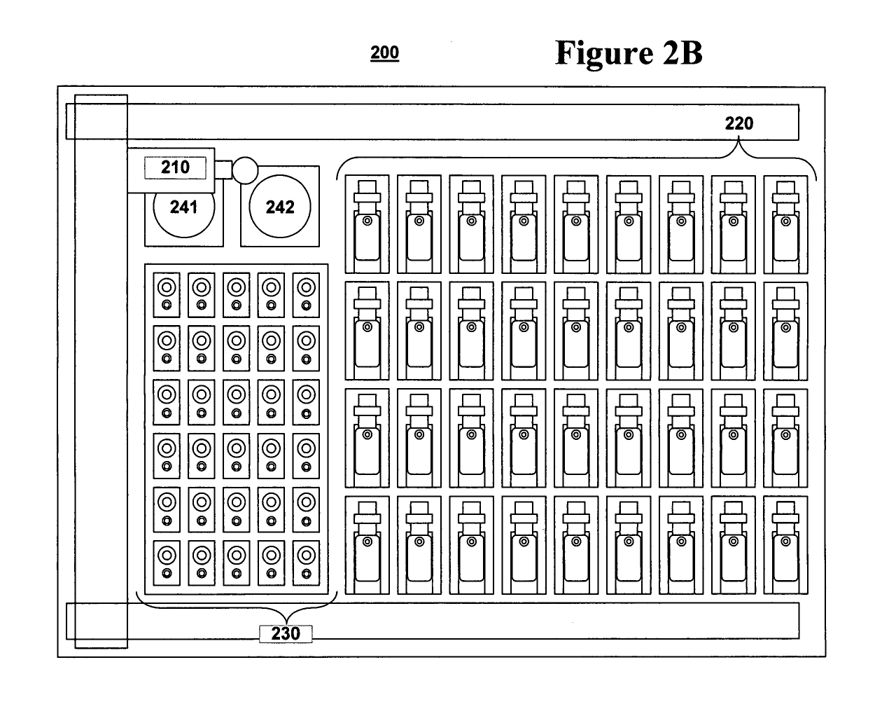

FIG. 2A illustrates a top view of first embodiment of a slide processing apparatus 200 of the present disclosure. The slide processing apparatus 200 is used for processing tissue specimens that are placed onto glass slides with a defined set of slide processing steps. The slide processing steps may include the application of chemical reagents, rinses, heating, drying, etc. A set of specific ordered tissue specimen processing steps is referred to as a “protocol”.

The slide processing apparatus 200 may be used to perform immunohistochemistry (IHC) protocols. Immunohistochemistry refers to the process of localizing antigens (such as proteins) in cells of a tissue section exploiting the principle of antibodies binding specifically to antigens in biological tissues. The slide processing apparatus 200 may also be used to perform the more complex in situ hybridization (ISH) protocols. In situ hybridization uses a labeled complementary DNA or RNA strand to localize a specific DNA or RNA sequence in a portion or section of tissue (in situ). An in situ hybridization protocol generally requires careful preparation, includes several processing stages that must be performed accurately, and may take several days to complete such that in situ hybridization is ideal for robotic processing.

The main area of the slide processing apparatus 200 is a slide module area 220 that contains a set of individual tissue slide modules. In the particular embodiment of a slide processing apparatus 200 illustrated in FIG. 2A there are three rows of slide modules wherein each row contains twelve slide modules for a total of thirty-six slide modules. However, this is only one example embodiment. Different implementations may contain a greater number or a lesser number of slide modules. For example, FIG. 2B illustrates an alternate embodiment with four rows of nine slides modules. All of the slide modules of the slide processing apparatus 200 are precisely arranged in a physical pattern that is programmed into the control system such that the slide processing apparatus 200 knows exactly where each slide module is located.

Adjacent to the slide module area 220 is a reagent container storage area 230. The reagent container storage area 230 stores a plurality of reagent containers that contain the chemical reagents that will be used to process the tissue specimen slides in the slide module area 220. Like the slide modules, the reagent containers in the reagent container storage area 230 are arranged in a physical pattern that is programmed into the control system such that the slide processing apparatus 200 knows the locations of all the reagent containers. Note that there is no specific arrangement required for the different chemical reagents, the slide processing apparatus 200 just needs to know all the reagent container positions in order to be able to accurately insert a probe into the opening of the reagent containers.

The slide processing apparatus also includes an area for storing a buffer solution container 241 and a waste container 242. In one embodiment, the solution container is very near the side of the apparatus so that it can easily be removed from the side of the apparatus. In other embodiments, the buffer solution container and/or the waste container are stored externally such that larger containers may be used for buffer solution and waste storage. However, the techniques presented in this disclosure allow the slide processing apparatus 200 to operate so efficiently that much less waste is generated than in prior art systems.

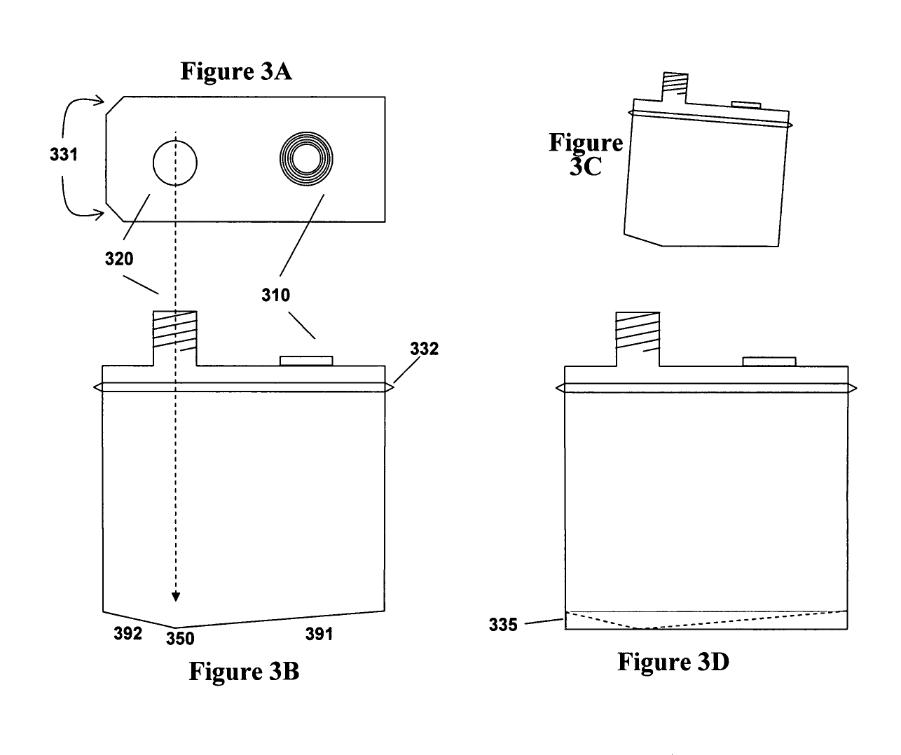

FIGS. 3A to 3D illustrate one embodiment of a reagent container that may be used within the reagent container storage area 230. FIG. 3A illustrates a top down view of one of the reagent containers. The reagent container has a relatively small opening 320 where a probe may enter to retrieve some of the reagent from the reagent container. The precision of the robotic arm used in the slide processing apparatus allows the opening 320 to be small. The opening 320 could be as small as a ten-thousandth of an inch in diameter. Having a small opening reduces the amount of reagent that will be lost due to evaporation. The reagent container may be “keyed” by having a designated shape (using the indentations 331 in the example of FIG. 3A) that only allows the reagent bottles to be placed in the proper orientation.

In addition to the container opening 320, the top of the reagent container may also have an associated Radio Frequency IDentifier (RFID). The RFID tag 310 on the reagent container allows detailed information about the contents of the reagent container to be stored on the RFID tag 310. The information initially stored on the RFID tag 310 may include the manufacturer identity, the type of chemical reagent, the volume of the container, and the concentration level as well as other information. Additional information may be written onto the RFID tag 310 by the slide processing apparatus. For example, the slide processing apparatus may write the date that a reagent bottle was first opened onto the RFID tag 320 so the slide processing apparatus can determine if expired chemical reagent is being used. Additional details about the use of the RFID tag 310 on a reagent container will be presented in a later section of this document.

Instead of the RFID tag 310 or in addition to the RFID tag 310, reagent containers may include barcode tags. Barcode systems are already used with other lab systems such that the use of barcodes would allow the disclosed system to integrate with other existing lab equipment. Thus, the same barcode labels for various chemical reagents may be used.

FIG. 3B illustrates a side view of one embodiment of a reagent container that may be used within the reagent container storage area 230. The reagent container of FIG. 3B has a ridge 332 that is used to suspend the reagent container when it is placed within a reagent container opening in the slide processing apparatus. The ridge 332 will hold the reagent container level such that the reagent probe may easily enter the opening 320 and proceed to the bottom of the reagent container 320. By suspending the reagent container in a reagent container rack, air is allowed to freely flow around the reagent container. This helps prevent the reagent containers from heating up due to a relatively close proximity with the slide modules that include a heating element for heat-treating slide specimens. By avoiding excess heat build-up, the costly chemical reagents will not be lost to evaporation or damaged due to the heat.

The bottom of the reagent container has two sloped bottom portions: a long sloped bottom portion 391 and a short sloped bottom portion 392. The two sloped bottom portions meet at an apex 350. This allows the last remaining drops of reagent in a nearly empty reagent container to collect at the apex 350. The robotic arm can place the reagent probe all the way down to the apex 350 such that nearly all of the chemical reagent in a container will be used, thus improving efficiency.

The shape of the reagent container also provides another convenience. Namely, when the reagent container is not within a reagent container storage area 230, the reagent container can stand stable on the long sloped bottom portion 391 as illustrated in FIG. 3C. In an alternate embodiment illustrated in FIG. 3D, the reagent container has an outside wall 335 that allows the reagent container to stand vertically while maintaining the “V” bottom for optimum fluid extraction.

Referring back to the FIG. 2A, the slide processing apparatus 200 includes a robotic arm that is capable of moving to any position above the reagent container storage area 230, the slide module area 220, and a “home” area (where the head 210 of the robotic arm is illustrated in FIG. 2A). The home area situates the head 210 of the robotic arm in a position that allows a technician to easily access the reagent container storage area 230 and the slide module area 220 in order to prepare the chemical reagents and tissue slides for a processing run. The robotic arm of the slide processing apparatus 200 is capable of precise movements in all three dimensions (X, Y, and Z).

The head assembly 210 of the robotic arm includes some equipment needed to process the tissue specimen slides. In one embodiment, the head assembly 210 of the robotic arm includes two different probes for handling liquids. Each probe ends with a vacuum controlled narrow tube for obtaining and depositing liquids. An in-line buffer solution supply is coupled to both probes to allow the probes to be cleaned by excreting buffer solution through the probe.

A first probe is used primarily for entering reagent containers and obtaining reagent fluids to be deposited on specimen slides. The first probe may be cleaned after each different reagent is used in order to prevent contamination between different chemical reagents.

A second probe is used to extract used reagent or buffer solution from slide modules. The extracted reagent or buffer solution is transported to the waste container 242. The second probe is also used to dispense buffer solution into the slide module using the in-line buffer solution from the buffer solution container 241. The first probe may also be used to dispense buffer solution but is not used for this purpose in one embodiment to minimize contamination.

In one embodiment, the head assembly 210 of the robotic arm includes a Radio Frequency IDentifier (RFID) sensor and writer unit. The Radio Frequency IDentifier (RFID) sensor/writer on the head 210 of the robotic arm is cable of reading from and writing to RFID tags placed on the reagent containers, slide modules, and/or the individual tissue specimen slides. This RFID sensor/writer capability provides significant information tracking abilities to the slide processing apparatus 200 that will be fully described in a later section of this document.

In one embodiment, the head 210 of the robotic arm includes a small digital image sensor. In this manner, the head 210 of the robotic arm can capture an image of a slide that the slide processing apparatus is processing. In particular, the image sensor may capture an image of the label area of a slide such that the slide processing apparatus 200 may store a digital image of the slide label along with the slide preparation protocol used when processing that particular slide. This provides a system for later verifying the specific processing protocol that was performed on a specific slide.

The digital image sensor may also be used for other purposes. For example, the digital image sensor can be used to capture an image of the tissue on the slide to determine the location of the tissue, the shape of the tissue, the size of the tissue, and whether the tissue is being stained. The digital image sensor may also be used to monitor slides during the slide processing. For example, the digital image sensor may be used to take an image of the specimen area to check for adequate liquid coverage or whether the reagent liquid is boiling or not.

A computer system (such as the computer system 100 of FIG. 1) may be used to run a slide processing apparatus control program that controls the slide processing apparatus 200 of FIG. 2A. Referring back to FIG. 1, the computer system 100 may be a standard personal computer system that sends control commands through a digital interface 130 to the slide processing apparatus. In one embodiment, the computer system 100 is coupled to the slide processing apparatus 200 using a standard Universal Serial Bus (USB) connection. The computer system may control the movement of the robotic arm, control the use of fluids by the probes on the head of the robotic arm, control the reading/writing with the RFID sensor, send commands to the individual slide modules, and perform various other operations in conjunction with the slide processing apparatus.

Various interface mechanisms may be used to control the computer system that controls the slide processing apparatus 200. In one embodiment, the computer system employs a touch-screen display and input device that allows users to select graphical images of slide modules on a slide matrix displayed on the touch-screen. The computer system may also be coupled to various wireless devices such that an operator may receive status and make changes remotely. For example, a tablet device such as an Apple iPad may be used to transmit commands to the computer system. The computer system that controls the slide processing apparatus 200 may also be provided with email address, cellular telephone number, or other electronic contact means of lab technician such that the computer system may transmit a message to the lab technician if a problem were to occur after a slide processing run has commenced execution.

Individual Slide Module Aspects

As illustrated in FIG. 2A, the slide processing apparatus 200 has a slide module area 220 that contains many individual slide processing modules. Each slide processing module holds an individual glass slide for processing. Each slide processing module may be an individual assembly that can be removed from the slide processing apparatus 200. In this manner, an individual slide processing module may be removed and replaced if that particular slide processing module has malfunctioned.

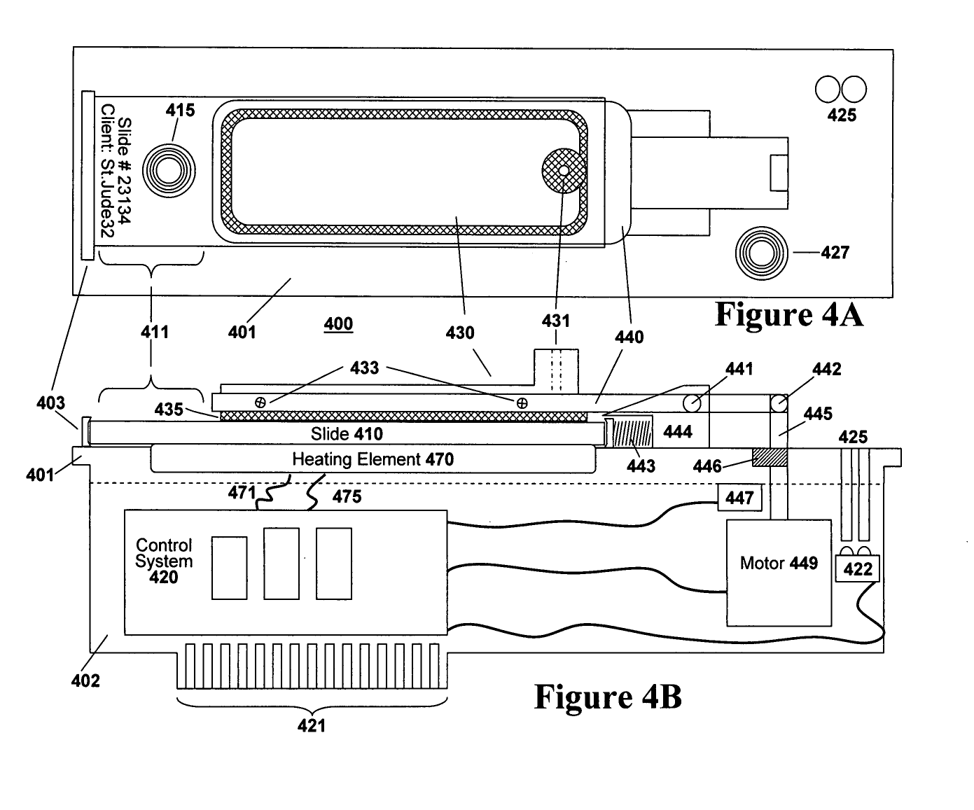

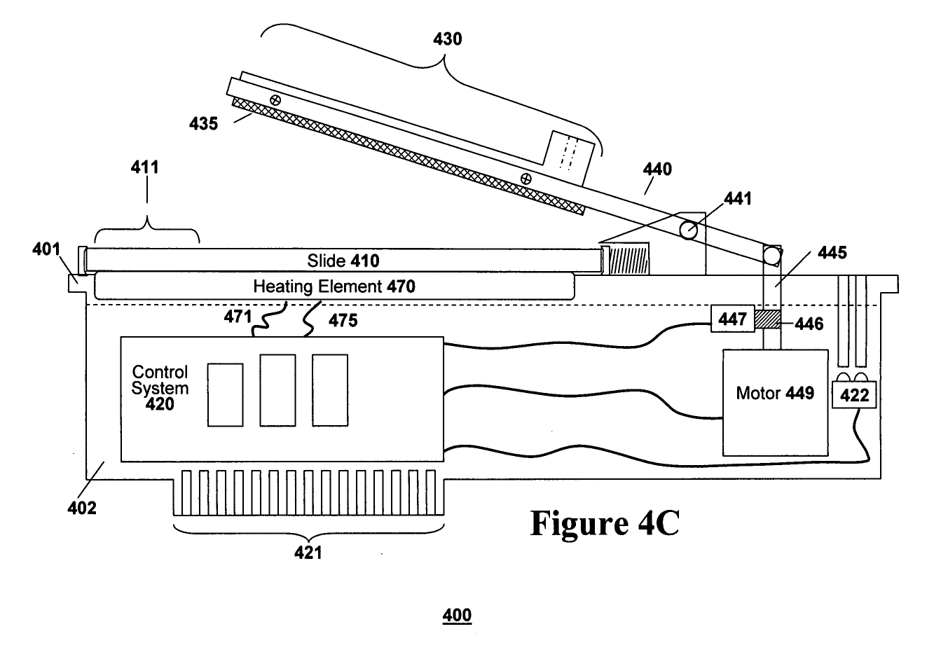

FIG. 4A illustrates a top view of one embodiment of a slide processing module 400. FIGS. 4B and 4C illustrate a side view of a slide processing module 400 with a lever unit 440 in a down and up position, respectively. The main structural components of a slide processing module 400 are a base structure 401 and a printed circuit board 402.

The base structure 401 is shaped to fit securely within an opening in the slide module area 220 for a slide processing module 400. Extending perpendicularly downward from the base structure 401 is a printed circuit board 402 as illustrated in FIG. 4B. The printed circuit board 402 has an interface 421 for electrically coupling with a main printed circuit board beneath the slide module area 220 of the slide processing apparatus 200.

In one embodiment, the printed circuit board 402 includes a local digital control system 420 (illustrated conceptually) that allows the slide processing module 400 to receive commands from the slide processing apparatus 200 and then independently execute those commands locally. In an alternate embodiment, the printed circuit board 402 lacks an independent control system such that the slide processing module 400 is controlled by a central control system for the slide processing apparatus 200.

The slide processing module 400 accepts a slide 410 that rests upon a heating element 470. The heating element 470 is carefully shimmed to be parallel with the base structure 401. Thus, when the lever unit 440 is closed down upon a glass slide 410 resting on the heating element 470, the lever unit 440 is seated substantially parallel to both the glass slide 410 and the base structure 401 that the lever unit 440 is mounted on. This ensures that the reagent fluid on the glass slide 410 will be sealed inside of a chamber formed using gasket 435. The flexible nature of the gasket 435 allows the slide module 400 to handle many different glass slide thicknesses that are within a defined tolerance range.

The glass slide 410 is inserted into slide processing module 400 by pushing one end of the glass slide 410 into a spring mechanism 443 within the hinge block 444 and then placing the other end of the glass slide 410 within a catch 403 at the front. The spring mechanism 443 pushes the glass slide 410 forward thus securing the glass slide 410 within catch 403 at the front.

The lever unit 440 is positioned partially above the glass slide 410 and extends beyond the end of the slide 410, through fulcrum 441 in hinge block 444, and on to a connecting pin 442. The lever unit 440 rotates about a fulcrum 441 allowing a first side of the lever unit to rise above the slide 410 and move down onto the glass slide 410. FIG. 4B illustrates the lever unit 440 in a down (closed) position where the lever unit 440 is situated parallel to and just slightly above the slide 410. In the disclosed embodiment, the slide processing module 400 uses a stepper motor 449 to rotate the lever unit 440 about fulcrum 441 by controlling a push rod 445 coupled by a connecting pin 442 at the end of the lever unit 440. FIG. 4C illustrates the lever unit 440 in an up position after the push rod 445 has been pulled down by stepper motor 449.

In the region of the lever unit 440 above the glass slide 410 there is an opening in the lever unit 440 for accepting a replaceable chamber unit 430. In one embodiment, the replaceable chamber unit 430 is held in place with a ball and spring plungers 433. The chamber unit 430 is used to contact the glass slide 410 with a gasket 435 and thus form a thin enclosed chamber above the slide 410. By having the chamber unit 430 as a replaceable unit (that is held in with ball and spring plungers 433 in one embodiment), an old chamber unit 430 can easily be replaced when the gasket 435 of the old chamber unit 430 is no longer able to form a tight seal with the top of the glass slide 410.

When the lever unit 440 is in the down position, down on the glass slide 410 as illustrated in FIG. 4B, the chamber unit 430 forms a sealed chamber on the top surface of the glass slide 410 that can be used to hold chemical reagents in place. When the lever unit 440 is in the down position the chamber only has a single opening 431 that is on the top of the chamber. However, the seal makes it difficult to introduce chemical reagents into the chamber. Thus, the local control system may lower the lever unit 440 down to an “injection position” that is slightly raised from the glass slide 410 such that there is a small second opening to the chamber above the slide 410 near the label area 411. After being placed in the injection position, the reagent dispensing probe on the head of the robotic arm may be inserted into a pliable opening 431 on the top of the chamber unit 430 to inject a chemical reagent. Due to the injection position of the lever unit 440, the injected chemical reagent fluid will flow from the area of the thin chamber near the pliable opening 431 toward the opening near the label area 411 thus spreading the chemical reagent across the entire specimen area of the glass slide 410.

After injecting the chemical reagent, the lever unit 440 may be lowered to the fully down position (as illustrated in FIG. 4B) to trap the reagent in a chamber formed by the gasket 435 sealed against the slide 410. The thin chamber above the glass slide 410 forces the chemical reagent to be distributed across the top surface of the glass slide 410. Furthermore, when the lever unit 440 is in the fully down position, the chemical reagent is not exposed to the surrounding air and thus cannot evaporate easily.

To remove chemical reagents, there is an “extraction position” for the lever unit 440. The extraction position will generally be similar to the injection position (and may actually be the same position in some embodiments). In one embodiment, the extraction position lifts the lever unit 440 slightly higher than the injection position. That higher extraction position allows liquid chemical reagent that is stuck to the top of the chamber unit 430 to move from near the label area 411 down toward the pliable opening 431 such that the chemical reagent may be extracted at the pliable opening 431.

The use of thin chamber above the slide specimen for handling chemical reagents applied to a glass slide provides several advantages to the slide processing apparatus. The thin chamber efficiently spreads the chemical reagent across the entire specimen area of the glass slide such that the entire specimen will be fully exposed to the chemical reagent. Using a thin chamber also allows a very small amount of chemical reagent to fully process each slide since the reagent will be spread across the entire specimen area of the glass slide. Therefore, the costs for chemical reagents will be minimized for the user of the disclosed slide processing apparatus. Furthermore, using a small amount of chemical reagent also minimizes the amount of waste fluid generated by the system. Since the chemical reagent fluids may be toxic, minimizing the amount of waste provides environmental benefits and minimizes waste disposal costs.

The length of the push rod 445 may be adjusted to achieve the proper pressure and seal when pressing the lever unit 440 and chamber unit 430 down onto the slide 410. An adjustable flag 446 may be attached to the push rod 445 in order to calibrate an open “home” position for the lever unit 440. Specifically, an optical sensor 447 is triggered when the flag 446 pass through the optical sensor 447. In this manner, the slide module 400 can always reset itself to a known “home” position by pulling down the push rod 445 until the flag 446 triggers the optical sensor 447. At that point, the slide module will know that it is in the open (“home”) position depicted in FIG. 4C.

To obtain a good seal between the gasket 435 of the chamber unit 430 and the glass slide 410, the control system 420 may ‘overdrive’ the stepper motor 449 until the stepper motor 449 slips. Repeatedly performing this might cause the system to become out of calibration. Having the flag 446 pass and the optical sensor 447 allows the system to recalibrate itself.

To further distribute a chemical reagent across the specimen area of the glass slide 410, the stepper motor 449 can be instructed to move the push rod 445 up and down several times such that the chamber unit 430 agitates the chemical reagent and spreads it across the top surface of the slide 410 with a rocking motion. Specifically, when chamber unit 430 presses down upon the slide 410, the chemical reagent will be moved from the end of the chamber near the pliable opening 431 to the end of the chamber near the slide's identification label area 411. When the lever unit 440 lifts the chamber unit 430 back up, the reagent may be pulled back to the end of the glass slide 410 near the pliable opening 431 with a capillary like action. Repeatedly opening and closing the chamber unit 430 will ensure that chemical reagent is spread across the entire specimen area and mixed with the tissue specimen.

To operate optimally, the stepper motor 449 is very carefully controlled. In one embodiment, the control system uses a progressive deceleration of the stepper motor near the open and closed endpoints. For example, when closing the lever unit 440 down onto the slide 410 the stepper motor 449 will be carefully decelerated before the gasket 435 forms a seal with the slide 410. This will prevent having the any liquid chemical reagent splash out and thus keeps the chemical reagent within the chamber formed when the lever unit 440 is in the down position. Similarly, when the stepper motor 449 lifts the lever unit 440 up, it will slowly decelerate such that the liquid will gather properly near the pliable opening 431.

Slide Module Electrical Control

As set forth above, one embodiment of the slide processing module 400 includes a local control system 420 for controlling the slide processing module 400. Instructions from a centralized controller can be provided to the local control system 420 and the local control system 420 will execute the commands. For example, the local control system 420 can be instructed to move the push rod 445 up and down for a specified time period in order to spread a chemical reagent across the glass slide 410. Similarly, the local control system 420 can be instructed to carefully control a heating element 470 that is used to carefully heat the glass slide 410. Heating the glass slide 410 may be performed to accomplish a variety of biological and chemical processes.

Each slide processing module 400 may be an individually addressable component accessible by a master computer system that is used to control the operation of the slide processing apparatus. In one embodiment each individual slide processing module 400 is handled as a separate a networked node on a single RS-485 serial bus. The master computer system thus communicates with each slide processing module 400 through the RS-485 serial bus. In one embodiment, the master computer system is coupled to the RS-485 serial bus through a USB to RS-485 protocol converter such that a common Universal Serial Bus (USB) port on a personal computer system can be used to control the slide processing apparatus.

Each individual node on the RS-485 bus (and thus each individual slide module) has a unique address. In one embodiment, the unique RS-485 bus address is encoded for each interface on the main board of the slide processing apparatus by a set of printed circuit traces on the main board coupled to the main board interface that couples to the interface 421 on a processing module 400. Thus, when a processing module 400 is inserted into the slide processing apparatus and plugged into the main board, that slide processing module 400 is assigned a unique slide module address. Therefore each individual slide processing module 400 may be identical but when inserted into a slide processing apparatus, that slide processing module 400 is given a unique address correlated with its physical location in the slide processing apparatus.

In addition to controlling the stepper motor 449 and the heating element 470, the local control system 420 may provide output in the form of visual indicators on the slide processing module 400. In the embodiment of FIGS. 4A to 4C, the local control system 420 controls multi colored Light Emitting Diodes (LEDs) 422 that may output status information. In one embodiment, a single multi-colored status LED used although FIGS. 4B and 4C illustrate an alternate embodiment with two multi-colored status LEDs 422.

The each local control system 420 of each slide processing module 400 may perform a local Power-On Self Test (POST) that performs various tests on the slide processing module 400 to ensure that the slide processing module 400 is operating properly. If the local control system 420 detects a problem with the slide processing module 400, the local control system 420 may output a diagnostic code in the form of an LED color and blinking pattern.

In one embodiment, each local control system 420 has a watchdog timer that monitors the slide processing module to ensure it is operating properly. If the slide processing module is not operating properly, the local control system 420 may reset itself in an attempt to restart normal operation. Similarly, the master computer system will test each slide processing module 400 to ensure that each slide processing module is communicating properly. If the master computer system cannot properly communicate with a slide processing module 400, the master computer system will alert operator about the failure.

A problem with an individual slide processing module 400 may be remedied by a technician. In the case of significant failure, the entire slide processing module 400 may be removed from the slide processing apparatus for replacement. The slide processing apparatus may be allowed to function with a missing slide processing module 400 such that the slide processing module apparatus may continue to be used while attempting to obtain a replacement slide processing module 400. This design ensures maximum up-time for the slide processing apparatus even when an individual slide processing module 400 has malfunctioned. A “dummy” cover may be placed in the missing slide processing module location to protect the main board from dust or fluids from entering the empty slide processing module location.

During normal operation of the slide processing apparatus, the local control system 420 may use the LEDs 422 to output various status and operating information. For example, a red or blinking red indicator may be used to indicate that a slide processing module 400 is currently in a slide heating stage. A green indicator may be used to indicate that the slide processing module 400 is operating properly.

Slide Module Heating and Cooling Features

As illustrated in FIGS. 4B and 4C, the local control system 420 may be used to control a heating element 470 within each slide processing module 400. The heating element 470 may be coupled to the local control system 420 with both a control conductor 471 and a sensor conductor 475. The sensor conductor 475 is coupled to a thermistor that allows the local control system 420 to determine the current temperature of the glass slide 410. The heat sensor for each heating element 470 is calibrated before deployment in order to provide an accurate reading of the glass slide 410 temperature. If a problem is detected, a temperature measurement probe may be used to recalibrate the heating system of a slide processing module 400.

The control conductor 471 allows the local control system 420 to provide electrical current in order to increase the temperature of the heating element 470. By testing the output of the thermistor on the sensor conductor 475 and controlling the current provided on the control conductor 471, the local control system 420 can very accurately control the temperature of the tissue specimen slide 410. In one embodiment, the current on the control conductor 471 is controlled in a digital manner wherein electrical current is either provided or not provided. In such an embodiment, the local control system 420 may employ a pulse width modulation (PWM) system to control the output of the heating element 470. In alternate embodiments, the actual amount of electrical current on the control conductor 471 may be modulated.

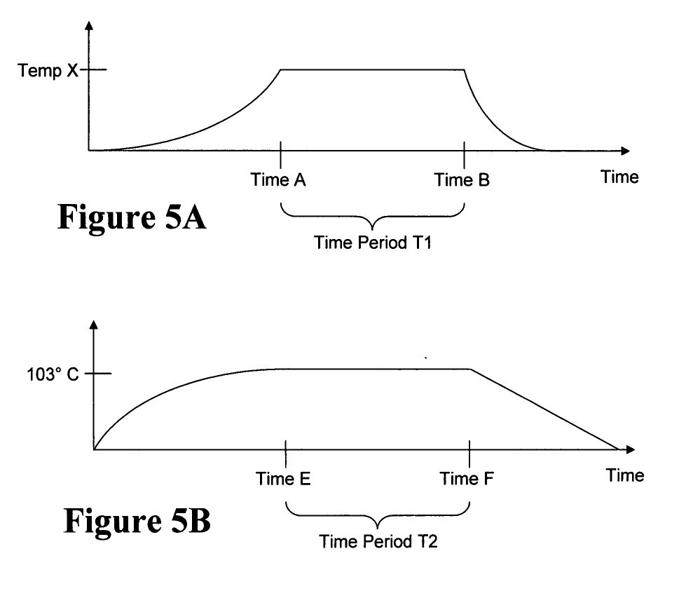

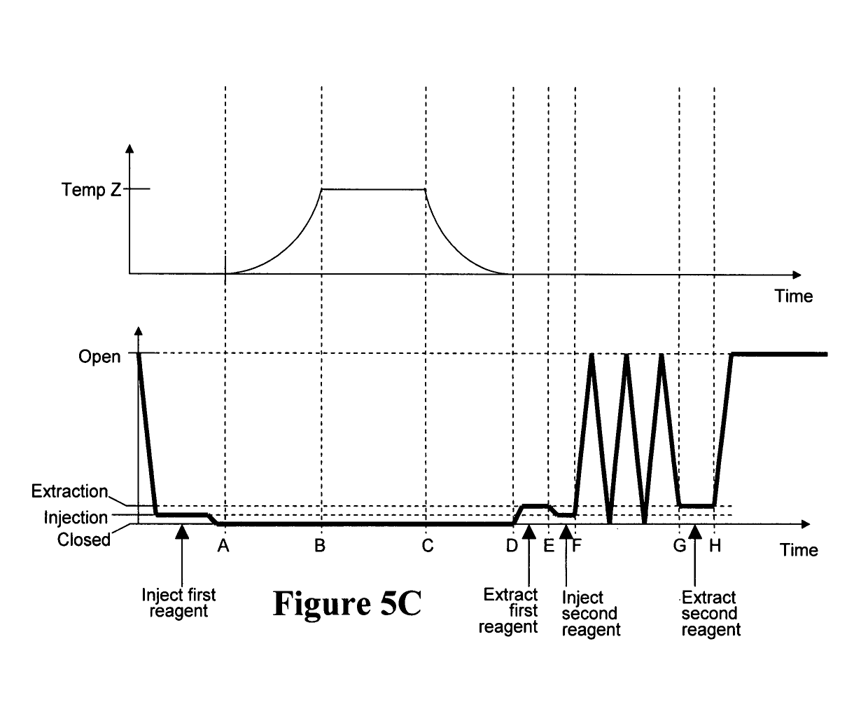

FIG. 5A illustrates a first example of one possible desired temperature control profile. In the desired temperature control profile of FIG. 5A, the system needs to slowly heat up the slide until the slide reaches temperature X at time A. Then, the temperature is to be held at temperature X for time period T1. Finally, the slide is allowed to be cool relatively quickly starting at time point B. To provide the heating up until time A, the local control system 420 can continually detect the current temperature using sensor wire 475 coupled to a thermistor and then control the current output on control wire 471 accordingly to follow the desired heating curve. During time period T1, the local control system 420 determines an appropriate on/off duty cycle for current output on control wire 471 to keep the tissue specimen slide 410 at the desired temperature. At time B, the control system enters a cooling stage. The local control system 420 modulates the current on control wire 471 to allow the slide 410 to cool at the desired rate.

FIG. 5B illustrates a second example of a possible temperature control profile that is made possible using teachings of the present disclosure. In the temperature control profile of FIG. 5B, the slide processing apparatus applies a reagent to a slide and then closes the chamber unit onto the tissue specimen slide. The local control system then heats up the slide with a concave curve until the slide reaches 103° C. at time E. In a traditional system, this might cause the much of the chemical reagent on the slide to evaporate since 103° C. is above the 100° C. boiling point of water. However, with the system of the present disclosure, the chamber unit keeps the reagent enclosed within the chamber unit such that the chemical reagent cannot easily evaporate. Thus, the disclosed system can keep the tissue specimen at 103° C. for time period T2. Finally, the slide is allowed to cool at a linear rate starting at time point F. If local control unit detects the slide cooling at faster than the linear rate, the local control unit may activate the heating element to keep the slide cooling at the linear rate specified by the protocol.

As long as the specified temperature rise rates and temperature fall rates are not too steep such that the temperature change rate exceed the limits of the heating system, the local control system 420 can be used to follow any specified temperature profile. For example, the heating and cooling stages may follow: linear, stepped, convex, concave, logarithmic, or other profiles. In one embodiment, the system allows a user to draw a heating/cooling curve and then use the data from that drawn curve to control the heating system. Having such precise temperature controls and the ability to heat above 100° C. will allow new slide processing protocols to be developed.

Again, it should be emphasized that each local control system 420 in each slide module 400 may be completely independent from the other control systems in other slide modules. In this manner, each local control system can be programmed with a different heating profile that will be carefully executed by that local control system. Furthermore, each local control system 420 can control the position of the chamber unit independently of all other slide modules. Since each local control system 420 controls both the local chamber unit and heating element, each local control system 420 can operate the local chamber unit and the heating element in a coordinated manner. An example of this coordinated activity is illustrated in FIG. 5C.

FIG. 5C graphically illustrates an example of coordinated action between the heating system and the chamber unit control system for a short protocol. The top graph depicts a heating control graph to be followed. The bottom graph illustrates a chamber control graph that depicts the position of the lever unit and the chamber unit. Initially, at the start of the protocol, the local control system of a slide module lowers the chamber unit down to the injection position just above the glass slide to allow a chemical reagent to be introduced. The slide processing apparatus then uses the robotic arm to inject a chemical reagent into the chamber unit. After introducing the chemical reagent, the local control system completely closes the chamber unit and begins to heat the specimen slide using heating system begins at time A. The local control system controls the heating element to heat the specimen slide following the heat control curve illustrated in the top graph from time A to time D.

At time D, the heating with a first chemical reagent is complete. Thus, the local control system lifts the chamber unit to the extraction position and extracts the first chemical reagent from the chamber unit. Next, at time E, the local control system moves the lever unit to the injection position and injects a new chemical reagent into the chamber unit. The protocol calls for the second chemical reagent to be agitated. Thus, the local control system agitates the second chemical reagent by successively raising and lowering the chamber unit onto the glass slide between time F and time G. At time G, the local control system lowers the chamber unit to the extraction position allow the second chemical reagent to be removed. At this point the slide processing protocol is complete such that the local control system raises the chamber unit at time H to allow the tissue specimen slide to be removed.

FIG. 5C illustrates only one simple example of countless different slide processing protocols that may be created with the disclosed system. Since each different slide processing module is individually controlled, each individual slide processing module 400 in the slide processing apparatus may execute a different protocol with different heating patterns and chamber unit control patterns. There are very few limitations such as the speed at which the slide may be heated or cooled and the fact that the different protocols being run on the same slide processing apparatus must allow enough time for the shared robotic arm system to deliver and extract chemical reagent fluids to the individual slide processing modules.

Radio Frequency Identifier Tag Usage

The slide processing apparatus 200 of the present disclosure may use Radio Frequency IDentifier (RFID) tags to improve the efficiency, accuracy, and data collection features of the slide processing apparatus 200. Referring to FIG. 2A, the head 210 of the robotic arm includes an RFID reader/writer. The RFID reader/writer can read data from and write data to reagent containers that have RFID tags attached, glass slides that have RFID tags attached, and slide modules 400 that have RFID tags attached. Each RFID tag is capable of storing a substantial amount of digital information.

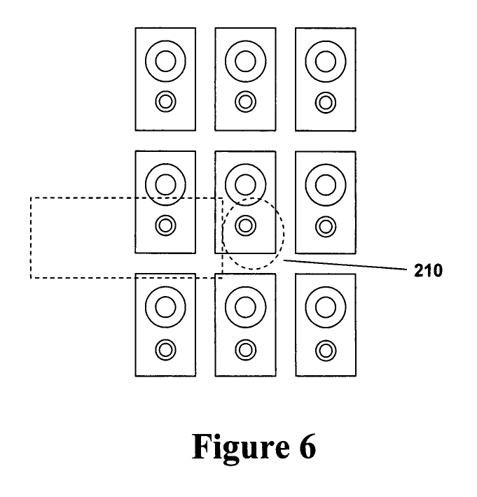

FIG. 6 conceptually illustrates the head assembly 210 over a set of reagent containers wherein each reagent container has a RFID tag. Since there are several RFID tags in close proximity to the head assembly 210, the RFID reader reads the signal strengths of the different RFID signals and selects the RFID signal with the strongest signal strength as the RFID tag associated with the reagent container that the head assembly 210 is above.

As set forth earlier, each reagent container may have an RFID tag that stores a substantial amount of information about the chemical reagent within the container. A set of static information that may be present is set forth in table 1.

Table 1—Example Static Reagent Container RFID Information

1) The type of chemical reagent stored within the container.

2) The concentration of the chemical reagent within the container.

3) The initial volume of chemical reagent within the container.

4) An expiration date for the chemical reagent within the container.

5) The identity of manufacturer that made the chemical reagent.

6) A specific lot number for the chemical reagent.

In addition to this static information, there may also be some dynamic information stored on the RFID tag of a reagent container. A set of dynamic information that may be stored on the reagent container is set forth in Table 2.

Table 2—Example Dynamic Reagent Container RFID Information

1) The time a reagent container was first used.

2) A running record of every time the reagent container was accessed.

3) An amount of reagent in the container using dead reckoning.

4) An amount of reagent in the container using probe measurement.

Note that since the system can measure the level of chemical reagent between subsequent uses of that chemical reagent, the slide processing apparatus receives an assurance that the chemical reagent is actually successfully being retrieved and deposited on a glass slide. This provides evidence that a glass slide was accurately processed using chemical reagents.

Between the ‘dead reckoning’ system of recording each extraction of chemical reagent and the actual measurement of reagent using the probe, the slide processing apparatus has two independent systems of keeping track of the chemical reagent volume within each reagent container. If these two chemical reagent volume values become out of synchronization by more than an expected error threshold, this disparity may be signaled to the operator of the slide processing apparatus.

There are several causes of such a volume disparity and it may be important to determine why there is a disparity. For example, a technician may have erroneously added some reagent to a reagent container in the reagent rack. This may not be advisable since the reagents may be different types, different concentrations, or may have other incompatibilities. Alternatively, if the difference occurs for most or all reagent containers then the calibration of the slide processing apparatus may need to be adjusted. Specifically, either the system for extracting a specific volume amount of chemical reagent or the probe's measuring system for measuring the volume amount of reagent within a reagent container may be out of calibration. Thus, when a difference occurs between the expected (from dead reckoning) and measured volumes for most or all reagent containers, then the slide processing system may need recalibration.

The RFID tags on the reagent containers provide a new manner of programming a slide processing apparatus and the master control computer for new slide processing protocols. For example, when a new chemical protocol is created for performing a new test on slide specimens, the entire details for the new slide processing protocol may be encoded onto the RFID tag of a new reagent container. The slide processing new protocol will specify all of the chemical reagent steps, the exposure times, any heating profiles, and any other protocol steps that are required for the new slide processing protocol. In this manner, the slide processing apparatus and the master computer system may be programmed with new slide processing protocols simply by adding a new reagent container (that is most likely used by the new protocol) into the reagent container storage area of the slide processing apparatus. The new slide processing protocol may then be selected when programming the slide processing apparatus for the next slide processing execution.

RFID tags may also be placed on individual glass slides. Referring back to FIGS. 4A and 4B, each glass slide 410 has an identification label area 411. The identification label area 411 may be used for storing written or printed identifier, an RFID tag 415, or both. An RFID tag 415 placed on the identification label area 411 may can be read by the RFID reader/writer and written on by the RFID reader/writer. There are numerous uses for the RFID tag 415 placed on the glass slide 410.

One particular usage is for the creation of glass slides with desired slide processing protocols pre-programmed onto the glass slide. Thus, when a person desires a particular slide processing protocol to be performed on a particular tissue specimen, the person can grab a glass slide that has been pre-programmed with an RFID tag that is programmed with a specific protocol to be performed. In this manner, the possibility of having a technician accidentally perform the wrong processing protocol performed is significantly reduced since the slide processing protocol will read the RFID to determine (or confirm) which slide processing protocol should be used on the slide.

Another usage for the RFID tag 415 placed on a glass slide 410 is detailed information tracking. Tissue specimen processing is often performed for very critical applications such as medical diagnostic tests and forensic criminal laboratory testing. For medical applications, it is critical to provide very accurate results since these medical diagnostic tests can include cancer biopsies that may literally be a matter of life and death for the patients being tested. For forensic criminal laboratory applications, it is also very critical to provide accurate results since a defendants life may be at stake. Forensic criminal laboratory applications also require that a clean chain-of-custody be maintained. The chain of custody information may include identifiers specify the various entities that have handled the slide such as the entity that placed the specimen on the slide, the entity that performed the slide processing, and the entity that examined the slide to determine the results.

Thus, to improve the information tracking, the slide processing apparatus may write information onto the RFID tag 415 upon processing a glass slide 410. A large amount of information may be written onto the RFID tag 415 to provide a full record of the slide processing. Examples of information that may be written onto the RFID tag 415 upon processing a glass slide 410 may include are presented in Table 3.

Table 3—Example Slide RFID Information

1) Identification of the entity that place the specimen on the slide.

2) Identification of the laboratory that processed the slide.

3) Identification the specific slide processing apparatus used.

4) Model information about the slide processing apparatus used.

5) Timestamp information about when the slide was processed.

6) A detailed list of the specific slide processing protocol steps performed.

7) Information about the reagents used as set forth in Table 1.

8) Identifier information about the patient or criminal case.

9) Identifier information linking any digital images taken of the slide.

10) A private key signature from the lab or slide processing apparatus.

Once a slide has been processed and had the processing information written onto the RFID tag 415, the slide may then be returned to entity that ordered the slide processing in order for a trained person to inspect the slide tissue specimen. If any entity questions the processing that was performed on the tissue specimen slide, that entity can read the RFID tag on the glass slide to obtain the detailed tracking information that was written onto the tissue specimen slide. This detailed information tracking thus provides a detailed record that can be used to verify that a tissue specimen slide has been processed properly.

For added assurance, encryption technology can be used to protect the data written onto the RFID tag 415. For example, the facility that processed a particular slide may sign the data written onto the RFID tag 415 of the slide. Since no other entity can forge such a private key signature, one can be assured that the tissue specimen was processed by that specific processing facility and the slide was processed exactly as specified.

RFID tags may also be placed on the slide processing modules 400. For example, FIG. 4A illustrates an RFID tag 427 mounted on the top surface of a slide processing module 400. By placing an RFID tags on slide processing modules, the slide processing apparatus may keep track of information about each slide processing module 400. Examples of information that may be written onto the RFID tag 427 of a slide processing module 400 may include:

Table 4—Example Slide Module RFID Information

1) Serial number of the slide processing module.

2) Date that the slide processing module was first used.

3) The number of slides processed by the slide processing module.

4) The date when the chamber unit was last replaced.

5) The number of slides processed since the chamber unit was last replaced.

6) Any problems that have occurred with the slide processing module.

This information may be used for maintaining maintenance schedules for slide processing modules. For example, the chamber unit has a pliable gasket used to create a tight seal on the glass slide. After a certain number of uses, the chamber unit should be replaced to ensure that a good seal is obtained. Thus, the slide processing apparatus may remind a lab technician when a chamber unit needs to be replaced.

Dewaxing Procedures

As set forth the previous sections, the disclosed slide processing module 400 includes features for depositing chemical reagents on the slide 410, agitating deposited chemical reagents using the lever unit 440 with its gasket 435, and heating the slide 410 with heating element 470. This combination of slide processing features allows the disclosed slide processing module 400 to perform function such as dewaxing slides that have been coated with a wax layer to protect the tissue specimen.

Many different processing steps may be followed in a dewaxing procedure. In one particular dewaxing procedure, the system heats the glass slide, deposits a chemical reagent onto the glass slide, and then agitates the reagent by moving the lever unit 440 up and down. The combination of the heat being applied and the chemical reagent work to break down the wax coating.

In another dewaxing procedure, the system turns on heating element 470 and lowers lever unit 440 down onto the glass slide 410. The chamber unit 430 forms a sealed chamber on top of the slide using gasket 435. The combination of these two actions create a heated chamber on the top surface of the slide since heat is applied from below but that heat is not allowed to escape due to the sealed chamber. The heated chamber can be used to melt the tax off the slide.

Slide Processing System Operation

The slide processing apparatus of the present disclosure can largely be programmed and operated using a touch-screen display in conjunction with a graphical user interface (GUI) on the master computer system. The graphical user interface allows a user to select slide processing protocols for individual slide modules by selecting a slide module from a map of the slide module area and then selecting a desired slide processing protocol from a list of different protocols. If the user wishes to assign the same slide processing protocol to several slide processing modules, the user may use a gesture wherein the user drags a finger across a set of slide modules on the map of the slide module area displayed on the touch-screen display.

This combination of a graphical user interface and a touch-screen input system allows a technician to program the slide processing apparatus easily within a laboratory environment with out needing a cursor control device and a keyboard. This reduces the chance of spilling a chemical reagent on a keyboard. (However, a keyboard and cursor control device may still be present for occasional use when performing certain complex configuration tasks such as creating a new protocol or any task requiring alpha-numeric input.)

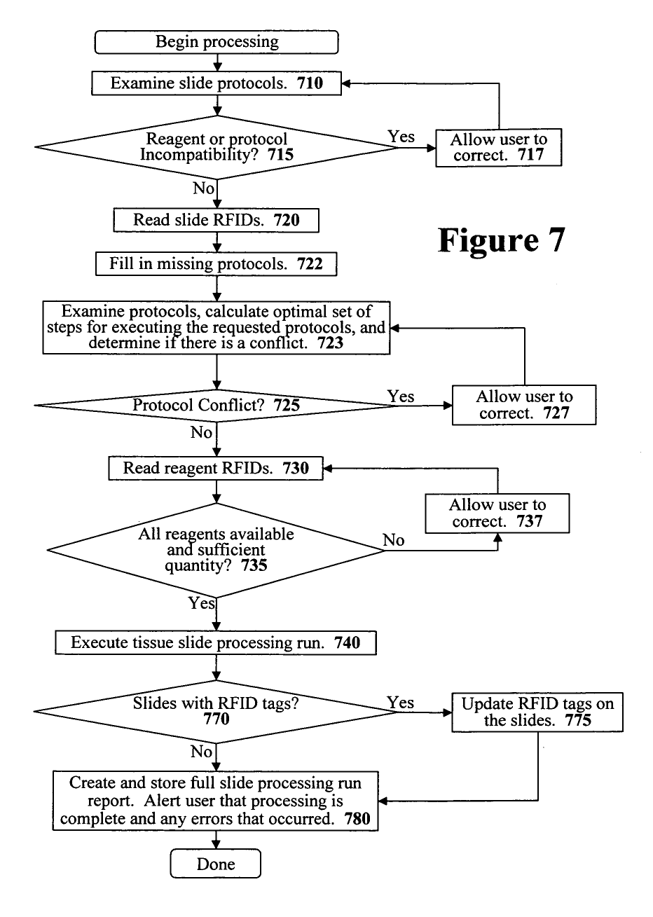

FIG. 7 illustrates a flow diagram that describes an example operation of the slide processing apparatus in one particular embodiment. Initially, a technician will fill the slide module area of the slide processing apparatus with glass slides containing tissue specimens to process. The technician will also enter specific processing protocols for those tissue specimen slides that do not have their own pre-programmed slide processing protocols on attached RFID. The technician must also fill the reagent container area of the slide processing module with all the chemical reagents needed to perform the specified slide processing steps. After these preparation steps, the technician then will then start the slide processing execution as depicted at the top of FIG. 7.

Initially, the master computer system that controls the slide processing apparatus will examine all of the slide processing protocols at stage 710 that have been entered by the technician. The computer system perform tests such as making sure that incompatible chemical reagent protocols are not used on the same tissue specimen. The system will check these slide processing steps and make decision at stage 715. If there are incompatibilities, the system will inform the technician and allow the technician to correct the discovered incompatibilities at stage 717 and then restart the test at stage 715.

The master computer system may then instruct the slide processing apparatus scan the slides in the slide module area to read any RFID tags on the slides at stage 720. (Digital images of each glass slide may also be taken at this time.) If any of the RFID tags on the slides specify a pre-programmed protocol to be performed then the master computer system will fill in those protocols at stage 722.

Next, the master computer system may again test the protocols at stage 725 for several factors. The master computer system will determine if the technician already entered a slide processing protocol for such a slide that specified a different slide processing protocol on the RFID tag such that the master computer system will allow the technician to select the proper protocol. The system may again test if the reagents of a pre-programmed protocol are incompatible such that the technician may correct the incompatibilities.

The master computer system will also perform a calculation to determine a set of robotic arm movements that will allow the slide processing apparatus to perform all of the specified slide processing protocols. Specifically, the master computer system must determine that there will be enough time for the robotic arm to make all the necessary movements to accurately perform the require steps (reagent deposition, reagent extraction, rinsing, etc.) for all of the specified slide processing protocols. The master computer system uses a set of proprietary algorithms that will determine the fastest way to execute the requested protocols. If there are any problems, the master computer system will allow the technician to make adjustments at stage 727 and then re-run the test at stage 725. For example, the technician may have to remove one or more tissue specimen slides if the slide processing apparatus is unable to determine a set of robotic arm movements that will fulfil all of the specified slide processing steps for all of the tissue specimen slides. However, this problem should only rarely occur.

The master computer system may then instruct the slide processing apparatus to scan the reagent containers in the reagent container area to read the RFID tags on the reagent containers at stage 730. The master computer system then determines whether all the necessary chemical reagents are available and have a sufficient volume amount of chemical reagent at stage 735.

At this point, the slide processing apparatus is ready to process the tissue specimen slides. Specifically, all of the slide modules that will be active have processing protocols assigned, all of the slide processing protocols are internally consistent, all of the different slide processing protocols are compatible in that the robotic arm will be able to perform all the needed movements to fulfil chemical processing steps, and all of the needed chemical reagents are available. All of these tests will ensure that once a slide processing run begins, it should be able to accurately process all of the slides. This is very important since a mistake during a slide processing run may damage a slide specimen. For example, if a chemical agent needed to stop the effects of a previous chemical reagent was missing or empty, that specimen may become over-exposed to the first chemical reagent and thus damaged.

After all of the preparation steps have been completed and the master computer system is satisfied that the slide processing run will execute without any problems, the slide processing apparatus then begins the slide processing run at stage 740. Although numerous tests were performed to ensure a reliable run, it is still possible for a problem to be encountered during the slide processing run. Thus, during the slide processing run, the slide processing apparatus may stop the slide processing run if a problem is detected and sound an alarm to alert a technician. Some of the problems that may cause the system to halt operation include running out of needed reagent unexpectedly, slide module failure,

If there is sufficient time to correct a problem detected during a slide processing run, the master computer system may allow a technician to correct the problem and then allow the slide processing to resume. If there is insufficient time to correct the problem that was encountered or there was no response from the technician after the alarm, the master computer system will attempt to complete the slide processing run as best it can. If a tissue specimen slide was not fully processed according to the requested slide processing protocol that will be reported to the technician. The slide processing problem may also be written onto the RFID tag of the glass slide that was not fully processed according to the requested slide processing protocol.

Once the slide processing run is completed, the slide processing apparatus will recall whether any tissue specimen slides had RFID tags at stage 770. If any tissue specimen slides did have RFID tags, then the slide processing apparatus will proceed to step 775. At step 775, the master computer system will instruct the slide processing apparatus to read the RFID tags of those tissue specimen slides, add the slide processing steps that were performed and other information from Table 2, and then write back the data onto the RFID tag of the tissue specimen slide.

The preceding technical disclosure is intended to be illustrative, and not restrictive. For example, the above-described embodiments (or one or more aspects thereof) may be used in combination with each other. Other embodiments will be apparent to those of skill in the art upon reviewing the above description. The scope of the claims should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim is still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. The abstract is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

We claim:

1. An automated biological specimen processing apparatus, said biological specimen processing apparatus comprising:
a biological specimen area, said biological specimen area containing a plurality of individual biological specimens, a first individual biological specimen of said plurality of individual biological specimens having a first RFID tag, said first RFID tag storing information associated with said first individual biological specimen; and
a robotic arm for depositing chemical reagents on said biological specimens and extracting chemical reagents from said biological specimens, said robotic having an RFID sensor for reading/writing said first RFID tag.

2. The biological specimen processing apparatus as set forth in claim 1 wherein said biological specimen processing apparatus reads a specific biological specimen processing protocol from said first individual biological specimen comprises and applies said specific biological specimen processing protocol to said first individual biological specimen.

3. The biological specimen processing apparatus as set forth in claim 2 wherein said biological specimen processing apparatus reads said set of processing steps from said first RFID tag and applies said set of processing steps to said first individual biological specimen.

4. The biological specimen processing apparatus as set forth in claim 1 wherein said information associated with said first individual biological specimen comprises a set of processing steps that have been applied to said first individual biological specimen.

5. The biological specimen processing apparatus as set forth in claim 1 wherein said information associated with said first individual biological specimen comprises an identifier associated with said first individual biological specimen.

6. The biological specimen processing apparatus as set forth in claim 1 wherein said information associated with said first individual biological specimen comprises an identifier associated with a laboratory using said biological specimen processing apparatus.

7. The biological specimen processing apparatus as set forth in claim 1 wherein said biological specimen processing apparatus stores information about said biological specimen processing apparatus onto said first RFID tag.

8. The biological specimen processing apparatus as set forth in claim 1 wherein said biological specimen processing apparatus stores a digital signature onto said first RFID tag.

9. The biological specimen processing apparatus as set forth in claim 1, said biological specimen processing apparatus further comprising:
a chemical reagent area, said chemical reagent area comprising a plurality of chemical reagent containers, a first chemical reagent container of said plurality of chemical reagent containers having a second RFID tag, said second RFID tag storing information associated with said first chemical reagent container.

10. The biological specimen processing apparatus as set forth in claim 9 wherein said information associated with said first chemical reagent container comprises the type of chemical reagent stored in said first chemical reagent container.

11. The biological specimen processing apparatus as set forth in claim 9 wherein said information associated with said first chemical reagent container comprises an amount of chemical reagent stored in said first chemical reagent container.

12. The biological specimen processing apparatus as set forth in claim 9 wherein said information associated with said first chemical reagent container comprises a biological specimen processing protocol that uses a chemical reagent stored in said first chemical reagent container, said biological specimen processing apparatus reading said biological specimen processing protocol to add said biological specimen processing protocol to a set of biological specimen processing protocols that said biological specimen processing apparatus may execute.

13. The biological specimen processing apparatus as set forth in claim 1, said biological specimen processing apparatus further comprising:
a plurality of biological specimen processing modules in said biological specimen area, each of said biological specimen processing modules including a biological specimen processing module RFID tag, said biological specimen processing module RFID tags storing information associated with the associated biological specimen processing module.

14. An automated biological specimen processing apparatus, said biological specimen processing apparatus comprising:
a biological specimen area, said biological specimen area containing a plurality of individual biological specimens; and
a chemical reagent area, said chemical reagent area comprising a plurality of chemical reagent containers, a first chemical reagent container of said plurality of chemical reagent containers having a first RFID tag, said first RFID tag storing information associated with said first chemical reagent container; and
a robotic arm for extracting chemical reagents from chemical reagent containers in said chemical reagent area and depositing chemical reagents on biological specimens in said biological specimen area, said robotic having an RFID sensor for reading/writing RFID tags such as said first RFID tag.

15. The biological specimen processing apparatus as set forth in claim 14 wherein said information associated with said first chemical reagent container comprises a type of chemical reagent stored in said first chemical reagent container.

16. The biological specimen processing apparatus as set forth in claim 15 wherein said information associated with said first chemical reagent container comprises a concentration of said type of chemical reagent stored in said first chemical reagent container.

17. The biological specimen processing apparatus as set forth in claim 14 wherein said information associated with said first chemical reagent container comprises an expiration date for a chemical reagent stored in said first chemical reagent container.

18. The biological specimen processing apparatus as set forth in claim 14 wherein said information associated with said first chemical reagent container comprises a time stamp.

19. The biological specimen processing apparatus as set forth in 14 wherein said information associated with said first chemical reagent container comprises an amount of chemical reagent stored in said first chemical reagent container.

20. The biological specimen processing apparatus as set forth in 19 wherein said amount of chemical reagent stored in said first chemical reagent container is computed using dead reckoning.

21. The biological specimen processing apparatus as set forth in 19 wherein said amount of chemical reagent stored in said first chemical reagent container is determined using a probe measurement.

22. The biological specimen processing apparatus as set forth in claim 14 wherein said information associated with said first chemical reagent container comprises a biological specimen processing protocol that uses a chemical reagent stored in said first chemical reagent container.

23. The biological specimen processing apparatus as set forth in claim 22 wherein said biological specimen processing apparatus reads said biological specimen processing protocol to add said biological specimen processing protocol to a set of biological specimen processing protocols that said biological specimen processing apparatus may execute.

24. A method of programming an automated biological specimen processing apparatus, said method comprising:
placing a biological specimen container in a biological specimen area of said automated biological specimen processing apparatus, said biological specimen container comprising an RFID tag, said RFID tag storing information specifying how a biological specimen associated with said biological specimen container should be processed;
reading said RFID tag on said biological specimen container using an RFID sensor on a robotic arm; and
programming said automated biological specimen processing apparatus using said information specifying how said biological specimen should be processed.

25. The method as set forth in claim 24 wherein said information specifying how said biological specimen should be processed comprises a slide processing protocol listing a set of steps and chemical reagents for processing said biological specimen.

26. The method as set forth in claim 24 wherein said RFID tag on said biological specimen container is preprogrammed for a specific biologic processing protocol.

27. A method of programming an automated biological specimen processing apparatus, said method comprising:
placing a biological specimen container in a biological specimen area of said automated biological specimen processing apparatus, said biological specimen container comprising an RFID tag;
processing a biological specimen associated with said biological specimen container with a specific biologic processing protocol;
reading said RFID tag on said biological specimen container using an RFID sensor on a robotic arm of said automated biological specimen processing apparatus to obtain said specific biological processing protocol; and
programming said automated biological specimen processing apparatus by adding said specific biological processing protocol to a set of biological processing protocols stored on said automated biological specimen processing apparatus.

28. The method as set forth in claim 24 wherein said specific biological processing protocol comprises a set of steps and chemical reagents for processing a biological specimen.

29. A method of tracking information with an automated biological specimen processing apparatus, said method comprising:
placing a biological specimen container in a biological specimen area of said automated biological specimen processing apparatus, said biological specimen container comprising an RFID tag;
processing a biological specimen associated with said biological specimen container with a specific biologic processing protocol;
writing information describing said specific biologic processing protocol onto said RFID tag on said biological specimen container using an RFID transceiver on a robotic arm of said automated biological specimen processing apparatus.

30. The method as set forth in claim 29 wherein said specific biological processing protocol comprises a set of steps and chemical reagents for processing a biological specimen.
